(19) United States
(12) Patent Application Publication
(10) Pub. No.: US 2006/0166266 A1
(43) Pub. Date: Jul. 27, 2006

(54) Screening method for regulatory enzymes employing tribrid (tri-hybrid) system

(76) Inventors: Alastair William Poole, Bristol (GB); Kanamarlapudi Venkateswarlu, Bristol (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/516,385

(22) PCT Filed: May 30, 2003
(86) PCT No.: PCT/GB03/02350

§ 371 Date: Jul. 13, 2005

(30) Foreign Application Priority Data
May 31, 2002 (GB) 0212652.2

(51) Int. Cl. C40B 40/10; C12N 15/74

(52) U.S. Cl. 435/7.1; 435/483

(57) ABSTRACT

A method for screening regulatory enzymes is described. The method involves the use of a tribrid cell engineered to express a DNA library.

This invention relates to a protein or polypeptide screening method. More particularly, the present invention relates to a method which identifies proteins or polypeptides having a defined function.

Proteins, or polypeptides, are important compounds within both a cell and the whole organism, whether the cell is from an animal, plant or is a micro-organism. Proteins are the key building blocks of both the cell and of the body with hugely diverse functions ranging from maintaining the structure of cells to the contraction of muscle, and for secretory proteins from the control of insulin secretion to the regulation of an immune response. To date, almost the full dictionary of the human genome has been determined but we are still a long way from knowing the full dictionary of the proteins encoded by these genes and are even further from understanding the functional significance of the proteins in the diverse range of different cell types present in the body. Additionally, the genomes of non-human animals or plants and of many important micro-organisms still await elucidation. For example, knowledge of protein or polypeptide function in the human, or other animal, may be useful in establishing the aetiology of disease, similarly in micro-organisms such knowledge would be useful in establishing the mechanism of infection of a pathogen. In plants, such knowledge would be useful in identifying and developing improved crop plants or for conservation of rare or endangered species.

The function of the majority of proteins is controlled by a wide range of changes to the protein, which changes are known as post-translational modifications. Generally, proteins tend to be either structural or globular (secretory) proteins according to the post-translational modification of the nascent protein into its tertiary or final conformation. These modifications may be transient or sustained, regulating protein function over short or long time spans. Modifications to proteins may be made by a huge range of enzymes (also proteins) which are at the heart of control mechanisms for cell function regardless of the origin of the cell. Many of these enzymes, especially human enzymes, are now characterised at the genomic level, but their precise function is still not known. Similarly, although details of some of the modifications that occur on proteins are known, the precise nature of the enzymes performing this regulation role is not clear. Yeast 2-hybrid was described as a method for investigating protein-protein interaction over 10 years ago. Since the initial report of the technique it has been modified and extended to allow screening of cDNA libraries to identify novel interacting proteins, and more recently it has been extended to the discovery of those protein-protein interactions which require ‘post-translational modification’. This has been most extensively applied to tyrosine phosphorylation-dependent protein-protein interaction, and because the interaction depends upon the insertion of a third gene, a tyrosine kinase, it has been called yeast tribrid. This yeast tribrid approach may also be used to screen cDNA libraries for protein-protein interactions, but in this case will only identify interacting proteins when the interaction depends upon tyrosine phosphorylation by the expressed third gene kinase.

The present invention is a novel extension of the yeast tribrid approach. In this the third gene itself is expressed as a cDNA library, and it is from this library that functional enzymes are identified. In other words the technology has the potential for identifying novel genes with a defined enzymatic function, rather than a physical association.

The prior art falls into two categories including methods relating to yeast hybrid approaches to identification of protein-protein interaction. In general, these rely upon identifying regulatory enzymes by detecting which protein-protein interaction their activity can induce. In some methods protein tyrosine phosphorylation is described as being a post-translational modification required for protein-protein interaction. For example, U.S. Pat. No. 6,242,183. describes a method of determining whether a first protein is capable of interacting with a second protein. The method involves the steps of a) providing a host cell which contains (i) a reporter gene operably linked to a protein binding site; (ii) a first fusion gene which expresses a first fusion protein, the first fusion protein including the first protein covalently bonded to a binding moiety which is capable of specifically binding to the protein binding site; and (iii) a second fusion gene which expresses a second fusion protein, the second fusion protein including the second protein covalenty bonded to a gene activating moiety and being conformationally constrained; and b) measuring expression of the reporter gene as a measure of an interaction between the first and second proteins.

Fields and Song ((1989) Nature 340, 245-246.) describe the first yeast two hybrid for studying protein-protein interaction. The second category concerns expression cloning of substrates of regulatory enzymes This method is described in several papers including a useful review article by Fashena et al (Gene 250 (2000) 1-14).

A problem with the methods of the prior art is that they have not addressed the identification of upstream signalling enzymes, but rather have addressed the identification of downstream substrates or interacting proteins. Thus when a substrate protein is identified, its activity and/or function still need to be determined.

It is therefore an object of the present invention to provide a functional screening method based on expression cloning which method is able to identify a comprehensive set of regulatory enzymes for any given cellular protein.

Accordingly, the present invention provides a screening method for regulatory enzymes, the method comprising the construction of a tribrid cell containing genes encoding an expression library of putative enzymes, a bait protein or polypeptide fused to a known-protein DNA binding domain and a prey protein which recognises a protein or polypeptide which has been post-translationally modified, the prey protein being attached to a known-protein active domain, whereby, in use, binding or recognition of the bait protein or polypeptide by the prey protein or polypeptide upon post-translational modification by an enzyme contained in the expression library, causes transcription of a reporter gene or genes which allow recognition of the enzymic activity.

Advantageously, the method of the present invention eliminates the need to further screening the identified protein or polypeptide for its function or activity and indeed is able to validate the function or activity of the protein or polypeptide without the need for further testing. This represents a substantial saving of time and effort by researchers in eliminating proteins or polypeptides which can be identified by a conventional method, but which do not have any activity or function or which do not have the required function or activity.

The cell may be eucaryote or a prokaryote. Preferably, the eucaryote cell is a yeast cell, although the use of cultured mammalian cells is intended to be included within the scope of the present invention.

By the term “tribrid” as used herein is intended a cell which has been engineered to express two proteins or polypeptides which are dependent on the activity of a third expressed protein or polypeptide for effect.

By the term “bait protein” as used herein is intended to describe any protein of interest and includes proteins of known, suspected and as-yet-unknown proteins which are or may prove to be important with regard to a diagnostic, therapeutic or pharmacological use. Preferred bait proteins include oncoproteins (such as myc, ras, src, fos and particularly the oligomeric interaction domains of fos) or any other proteins involved in cell cycle regulation such as, for example, kinases, phospatases, or the cytplasmic domains of receptor proteins. Examples of potential bait proteins include cyclin and cycin-dependent kinases (for example Cdk2), receptor-ligand pairs, neurotransmitter protein pairs, or other pairs of signalling proteins. In each case, the bait protein will be attached to a known nucleic acid binding domain.

By the term “prey protein” as used herein is intended a protein which is conformationally constrained preferably by being either embedded in a conformation-constraining protein or by linking the carboxy and amino termini of the protein. Optionally, the prey protein further comprises an epitope tag which enable rapid detection of fusion protein synthesis using conventional immunological techniques. Prey proteins other than those described herein may be useful in the present invention. For example, cDNAs may be constructed from any mRNA population and inserted into a suitable expression vector using well known commercially available kits and methods. Preferably, the prey protein of the present invention is a single chain antibody or other detector protein or polypeptide module such as the polypeptides known as SH2, PTB, 14-3-3, or WW domain.

Any enzyme may be used in the present invention but, preferably, the enzyme is an enzyme involved in post-translational modification of nascent proteins or polypeptides. More preferably, the enzyme has a role in the addition or removal of phosphorous, sugars, or sulphur, or in side chain extension (e.g. acetylation) or reduction or branching, including amidation, nitrosylation, ubiquitination, myristoylation and palmitoylation. Ideally, the invention identifies enzymes which have an association with or are responsible for the addition or removal of phosphorus to a protein or polypeptide, e.g. a kinase or a phosphatase. Of these the invention is particularly concerned with tyrosine and serine/threonine kinases or phosphatases, and the enzymes which are upstream or downstream in cascade systems involving a tyrosine or serine/threonine kinase or phosphatase.

In a second aspect, the invention provides a tribrid cell for use in the above described method.

Preferably the tribrid cell as hereinbefore described is a eucaryotic cell, especially a yeast cell.

Preferably, the tribrid cell is engineered to express a cDNA library of enzymes or putative enzymes, and a prey protein as hereinabove defined. The cell is also able to be transformed to receive the substrate or bait protein as hereinabove defined; when all the components (library, prey and bait) are expressed by the cell, the cell is a tribrid cell as hereinbefore defined and is suitable for use in the method of the invention.

Preferably interaction of the bait and prey proteins induces post-translational modification of a preselected protein expressed by the tribrid cell.

It is possible to detect this modified protein, for example by use of a marker reaction (such as a colourimetric reaction) or by the use of an

In a preferred embodiment of the invention, an antibody is used. Preferably, the antibody is a single chain antibody or a monoclonal antibody.

In the description which follows, the present invention will be described with reference to use in a yeast cell. However, it is to be understood that this is not intended to limit the scope of the invention since the method finds equal utility in any tribrid cell.

Similarly, the present invention will be described with reference to kinases, although it is not intended to limit the scope of the invention to kinases, since any enzyme may be used with equal utility.

Embodiments of the invention will now be described, by way of example only, with reference to the following accompanying drawings, of which:-

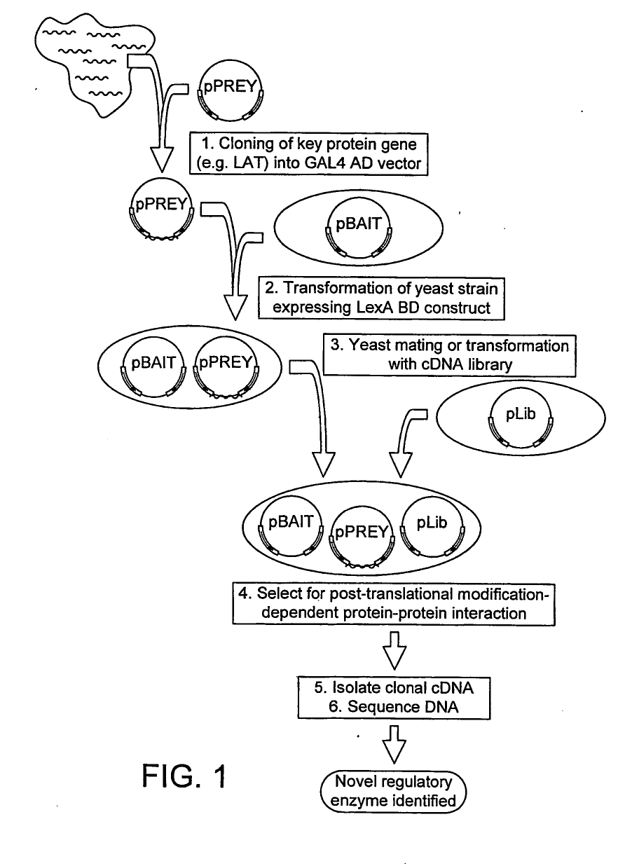

FIG. 1 is a diagram showing a summary of the method steps involved in the cloning of functional enzymes involved in the post-translational modification of proteins. LAT—Linker for activation of T-lymphocytes; pPREY—DNA binding domain fusion vector for genes to identify post-translational modifications; pBAIT—transcriptional activation domain fusion vector for gene of interest. Genes used to identify post-translational modifications, including SH2 and PTB domains for phosphotyrosine, and single chain antibodies for a broad spectrum of modifications; pLIB—cDNA library of post-translational modifying enzymes with or without nuclear localisation sequence fusion tag.

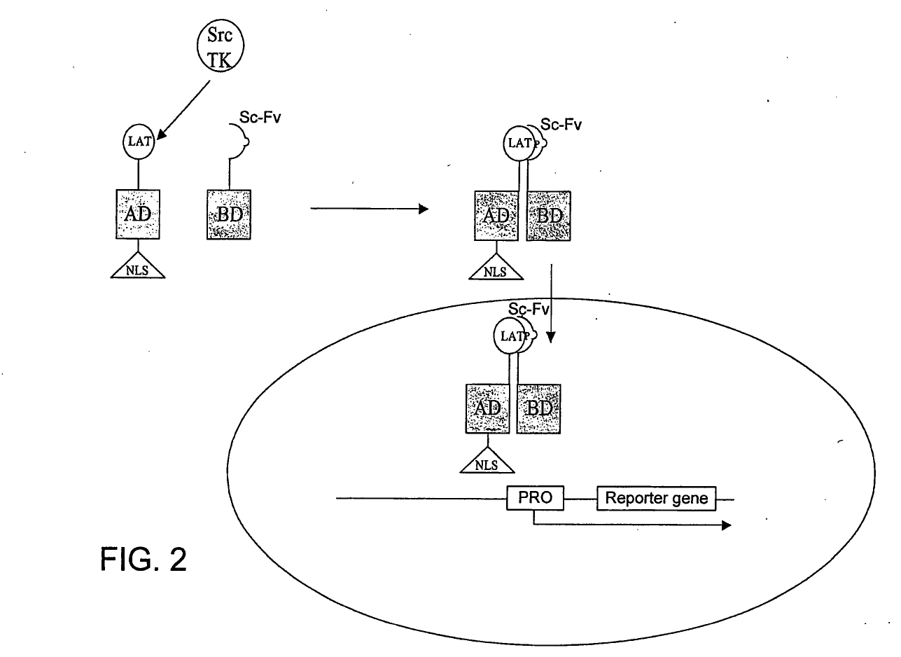

FIG. 2 is a diagram showing the tribrid approach to identification of Src as a kinase phosphorylating LAT, using Sc-Fv anti-phosphotyrosine antibody as prey. NLS—nuclear localisation sequence; AD—activation domain; BD—DNA binding domain; LAT—linker for activation of T-cells; Sc-Fv—variable region of single-chain antibody; P—phosphate; Pro—promoter region of gene; TK—tyrosine kinase domain.

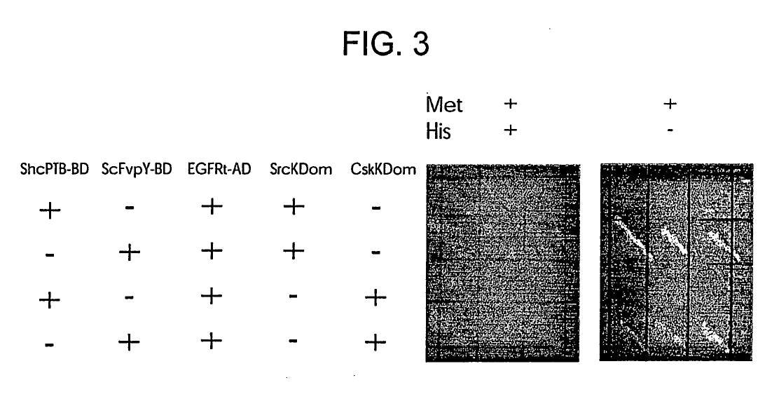

FIG. 3 shows HIS3 gene leakiness when ScFvpY fused to LexA DNA binding domain. ShcPTB-BD—phosphotyrosine binding domain (PTB) of Shc fused to LexA DNA biding domain; ScFvPY-BD—anti-phosphotyrosine single-chain antibody fused to LexA DNA binding domain; EGFRt-AD—C-terminal tail region of human EGF receptor fused to GAL4 activation domain; SrcKDom—Src active kinase domain; CskKDom—Csk active kinase domain.

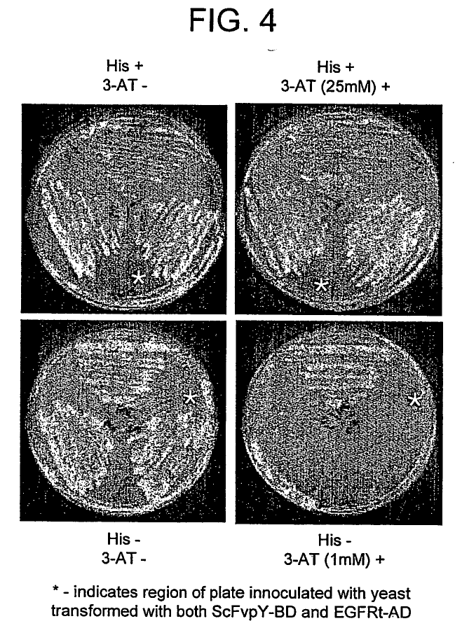

FIG. 4 is a series of photographs showing that 1 mM 3-amino-1,2,4-triazole overcomes leakiness of HIS3 gene expression when yeast are transformed with ScFvpY-BD and EGFRt-Ab. * indicates region of plate inoculated with yeast transformed with both ScFvpY-BD and EGFRt-AD.

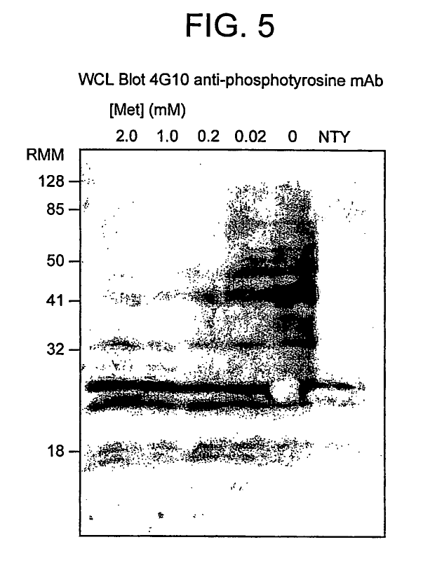

FIG. 5 shows a western blot of yeast whole cell lysates from yeast singly transformed with the Src kinase domain construct. NTY—non-transformed yeast; Met—methionine; WCL—whole cell lysate. This figure shows that methionine suppresses expression of active Src kinase domain in yeast transformed with this construct under the met-repressible promoter.

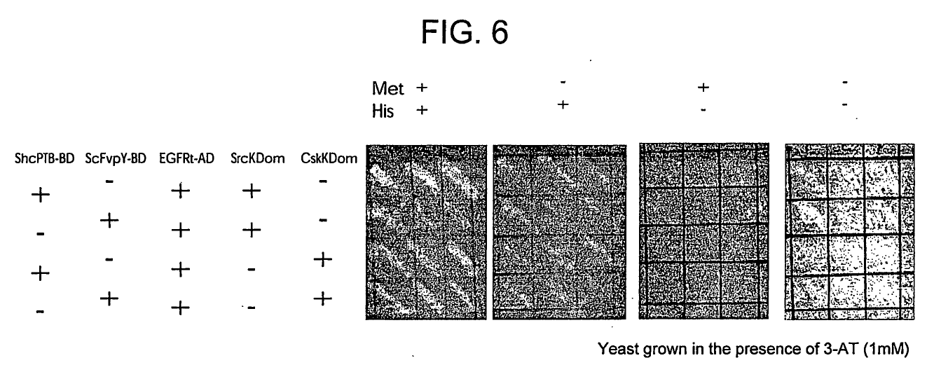

FIG. 6 shows the results of tribrid assay showing Src to phosphorylate EGF receptor tail region: trapping prey is either ShcPTB domain or a single chain anti-phosphotyrosine antibody (ScFvpY-BD).

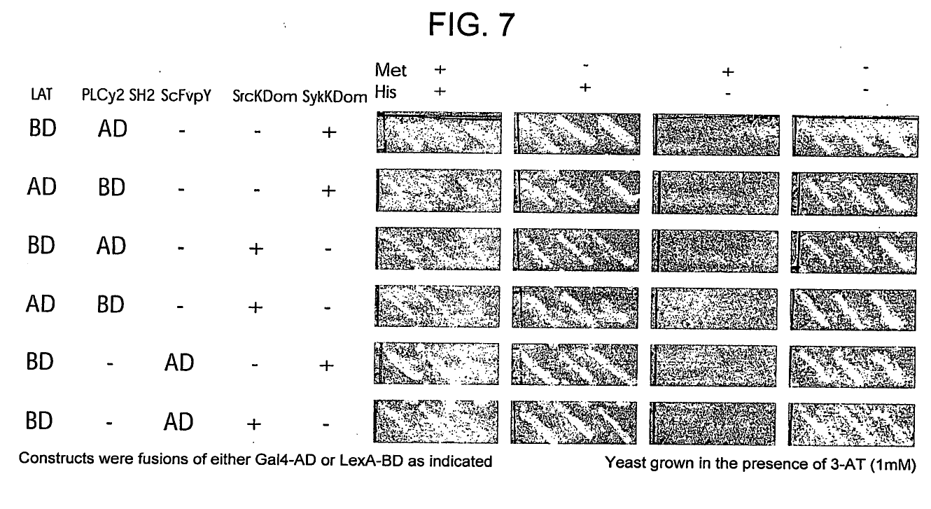

FIG. 7 shows the results of tribrid assay showing tyrosine phosphorylation of LAT by Src or Syk: phosphorylation detected by prey proteins PLCy2-SH2 domains or single-chain anti-phosphotyrosine (ScFvPY). LAT—linker for activation of T-lymphocytes; PLCy2 SH2—tandem Sh2 domains of PLCg2; ScFvpY—single-chain anti-phosphotyrosine antibody; SrcKDom—active catalytic domain of human tyrosine kinase Src; SykKDom—active catalytic domain of human tyrosine kinase Syk.

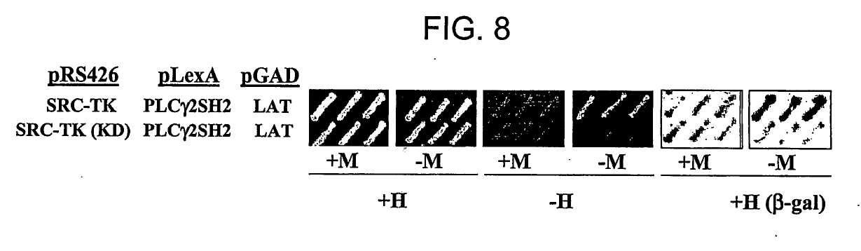

FIG. 8 shows the results of yeast tribrid assay demonstrating that non-catalytically active point mutant of Src is unable to sustain interaction between PLCy2-SH2 and LAT. The results show that LAT interacts with the PLCγ2SH2 upon phosphorylation by SRC in yeast tribrid assay. The pLexA- and the pGAD-fusion constructs were co-transformed with the pRS426-fusion constructs into TAT7 yeast strain and tested for their ability to grow in the absence of histidine and methionine and to express β-galactosidase, which was analysed by assaying the conversion of X-gal into a blue coloured product. The panel, left to right, shows 3 clones of each transformant grown on solid medium with and without histidine (−H and +H) and methionine (−M and +M) and filter β-galactosidase assay (β-gal). Src-TK—catalytically active kinase domain of human Src; Src-TK(KD)—catalytically inactive point mutant of the kinase domain of human Src.

### EXAMPLE 1

The inventors prepared a novel yeast three hybrid approach to screening a human Jurkat T-lymphocyte cDNA library for upstream tyrosine kinase regulators of LAT function. LAT (linker for activation of T-cells) plays a critical role in regulating T-cell activity and is the link from early kinase activity, especially ZAP70 and Syk, to activation of PLCY isoforms. LAT is highly tyrosine phosphorylated, and although the inventors know that members of the Syk family of tyrosine kinases is responsible for its phosphorylation, there are likely to be other, as yet unidentified, kinases which are also responsible for its phosphorylation and play a crucial role therefore in regulating T-cell activity. The system involves three proteins, which allow or prevent the formation of the transcriptional activator. Beside the two-hybrid fusion proteins, the third partner is expressed from the Jurkat cDNA library under the control of the Met25 partner. This gene is positively regulated in medium lacking methionine. Here we show a situation where specific tyrosine kinases encoded as the third partner in the assay, promote the interaction between two proteins, one fused to a DNA-binding domain and the other fused to an activator domain. In this way the Jurkat cDNA library was screened and a novel tyrosine kinase, kinase X, was identified which was subsequently demonstrated to be responsible for phosphorylating LAT in Jurkat T-cells.

Yeast Transformation

Saccharomyces cerevisiae strain TAT7 (MATA ade2, ura3, leu2, trp1, his1) was used for triple transformations. For histidine (HIS) auxotrophy assays, yeast were spotted on the appropriate drop out plates lacking HIS and supplemented with 10 mM 3-aminotriazole (3-AT). β-galactosidase assays were performed in liquid culture or on nitrocellulose filters as described below. General yeast methods were used as previously described.

β-Galactosidase Assays

For filter assays, diploids were streaked onto nitrocellulose filters and quantitative determinations of β-galactosidase activity were performed by standard methods. Briefly, diploids (10⁷ cells/ml) were pelleted and broken by vigorous shaking in 0.2 ml of breaking buffer (100 mM Tris-HCl, pH 8, 1 mM DTT, 20% glycerol) after addition of glass beads. 100 ml of cleared diploid lysate were incubated at 28° C. for 0.54 hours (T, in minutes) with 20 ml o-nitrophenyl β-D-galactopyranoside (ONPG, 1.5 mg/ml). The reaction was stopped by addition of 50 ml 1 M Na₂CO₃ and OD₄₀₅ was measured. Protein concentration (C, in mg/ml) in the cleared lysates was determined using the Biorad protein assay. β-galactosidase activity (A) expressed in nmol/min/mg was calculated according to the formula: A=1.70D₄₀₅/0.045TC, where 1.7 corrects for the reaction volume and 0.0045 is the optical density of a 1 nmol/ml solution of ONPG.

Plasmid Constructs

The yeast-E. coli shuttle plasmid YlexA, which contains a galactose-inducible promoter and transcription initiation and termination sequences, was used. Inserted into the polylinker is the gene for the E. coli protein LexA (amino acids 1-202) which binds to the Lex operator and restriction sites for cloning other genes in frame. This plasmid was digested with appropriate restriction enzymes and the following cDNAs were inserted to form ‘prey’ proteins: (i) human PLCγ2 tandem SH2 domains, pPLCSH2, or (ii) a single chain antiphosphotyrosine antibody, pYScFv, used a general detector to identify tyrosine phosphorylation multiple substrates. The bait protein substrate LAT gene was fused to the transcription activation domain for Gal4 to form plasmid pLAT.

Screening of a Human cDNA Library

A human Jurkat T-lymphocyte cDNA library, pLib, was constructed. Briefly the cDNA was synthesised from adult mouse brain poly(A)-selected mRNA using random and oligo(dT) primers. EcoRI linkers were attached to the cDNA. The cDNA was sized and fragments longer than 500 bp were inserted into EcoRI site of the appropriate vector. The primary library consists of I million independent colonies. For the library screening, TAT7 yeast harbouring pLAT and pPLCSH2 or pYScFv was transformed with 0.5 mg of the cDNA library plasmid (purified from bacterial culture, spread on the appropriate drop out plates lacking HIS and supplemented with 10 mM 3-AT and incubated at 30° C. for 7 days. His⁺ transformants were tested for β-galactosidase activity as described above. pLib plasmids were isolated from the β-galactosidase positive transformants and the insert was determined by standard DNA sequencing.

In order to validate the screening assay for novel kinases involved in tyrosine phosphorylation of LAT, Syk constructs were used to replace pLib. Syk is known to phosphorylate LAT and was used as a positive control for the screening assay.

In Vitro Phosphorylation of LAT

Kinase X, identified from the tribrid library screen, was expressed with a His tag in insect SF9 cells, and purified by standard procedures on Ni²⁺ columns. LAT was expressed as a GST-fusion in BL21 E. coli bacteria, and purified by standard procedures using glutathione-agarose beads. Lat-bound beads were suspended in 20 μl of kinase assay buffer (5 mM MgCl₂, 5 mM MnCl₂, 100 mM NaCl, 10 μM ATP, 20 mM HEPES at pH 7.2), mixed with 2 μg of kinase X or recombinant Syk as a positive control, or buffer as a negative control, and the reaction started by addition of γ-³²P]-ATP (250 μCi/ml). After incubation for 15 min at 25° C., the reaction was terminated by addition of ice cold EDTA (0.5 M). Beads were subsequently washed, lysed in sample buffer and proteins separated by SDS-PAGE, transferred to PVDF membrane and subjected to autoradiography for detection of autophosphorylation label. Membranes were subsequently immunoblotted (as described below) for detection of phosphotyrosine.

Generation of Dominant-Negative Mutant (DNM) Kinase X

Full length kinase X, identified from the tribrid library screen, was mutated at a critical residue within the kinase domain to generate a kinase-null mutant. Standard site-directed mutagenesis was used to generate this mutant. The kinase was otherwise intact, but lacked any kinase activity and was therefore suitable for use as a dominant-negative mutant. Constructs for kinase X and DNM kinase X were cloned into the mammalian expression vector pcDNA3. Jurkat T-cells were transfected with either of these constructs using a lipid-based transfection system. The role of kinase X in regulating phosphorylation of LAT and in regulating T-cell activity was then assessed.

Immunoprecipitation of LAT

Jurkat T-cells transfected with wild-type kinase X, DNM kinase X or empty vector as control, were stimulated by cross-linking of CD3. Reactions stopped by lysis with an equal volume of 2× extraction buffer (2% (v/v) Triton X-100, 300 mM NaCl, 20 mM Tris, 1 mM PMSF, 10 mM EDTA, 2 mM Na₃VO₄, 10 μg/ml leupeptin, 10 μg/ml aprotinin, 1 μg/ml pepstatin A, pH 7.3), and insoluble material removed by centrifugation (13000 g, 5 min, 4° C.). Supernatants were then pre-cleared by incubation with protein A-sepharose (PAS) for 1 hour at 4° C., followed by centrifugation (13000 g, 5 min, 4° C.). Supernatants were then incubated with PAS beads and anti-LAT antibody for 120 min at 4° C. Beads were then washed once in extraction buffer and a further twice in TBS-T before addition of Laemmli sample-treatment buffer. Precipitated proteins were then subjected to SDS-PAGE, transferred to PVDF membrane and probed with appropriate antibodies as described in “Immunoblotting” below.

Immunoblotting

Jurkat cells were activated by cross-linking of CD3 and reactions were stopped by adding an equal volume of Laemmli buffer (2×). Samples were heated for 5 min at 95° C. Proteins were separated by either 10% SDS-PAGE or by SDS-PAGE on 5-15% gradient slab gels and transferred to PVDF blotting membranes using a semi-dry transfer system (60 min, 15V). Membranes were incubated for 60 min at room temperature with primary followed by secondary antibodies and detected by ECL (Amersham, UK).

### EXAMPLE 2

FIGS. 1 & 2 shows the general approach being made to screen a cDNA library for functionally important genes which may regulate the activity of known important key disease-related proteins. For example, it is known that the protein LAT (linker for activation of T-cells) is a highly regulated scaffolding protein required in T lymphocytes to enable the T cell receptor to couple to major signalling and functional events, including the activation of phospholipase Cy isoforms, a cell calcium response and induction of IL2 gene transcription. Although it is known that LAT is highly tyrosine phosphorylated upon activation of T cells, and that this phosphorylation is required for downstream signalling events, it is as yet not clear exactly which tyrosine kinases are responsible for the phosphorylation events. This approach is able to screen a library of human kinases to identify putative upstream kinases, which may represent important novel targets in the treatment of T cell-mediated diseases.

Protein of interest, in this case LAT, is cloned into pPREY vector to generate a fusion construct with the GAL4 activation domain and transformed into yeast. PBAIT vectors are constructed such that they express fusions of LexA BD domains with domains that will selectively identify post-translational modifications. In the case of tyrosine phosphorylation, SH2 or phosphotyrosine binding (PTB) domains may be used, but importantly we have also used a selective single chain antibody (ScFvpY) to detect phosphorylation on tyrosine residues. Yeast is then doubly transformed with this construct plus the prey construct. A library of cDNAs encoding active (or inactive as controls) post-translational modifying enzymes is then transformed to form a triply transformed yeast. Markers genes will be selected for when the protein of interest is modified (in this case LAT phosphorylated on tyrosine) by enzymes expressed in the library.

In the following experiments two sets of kinase/substrate partners were assayed to demonstrate proof of concept and determine optimal conditions for the detector fusion protein to operate to detect post-translational modifications. Both sets centre on tyrosine phosphorylation: (i) uses the LAT system described above, where LAT is able to be phosphorylated by Syk and by members of the Src family of kinases. When phosphorylated on tyrosines, LAT then allows binding of PLCγ2 via its SH2 domains. Therefore for this system the detector proteins are the PLCγ2SH2 domains (tandem) and the anti-phosphotyrosine single-chain antibody ScFvpY. (ii) uses the cytoplasmic tail of the receptor for epidermal growth factor (EGF) as a known substrate for Src. When phosphorylated on tyrosine, the EGF receptor acts as a binding site for the phosphotyrosine binding domain (PTB) of Shc. This is therefore used in some assays as the detector protein. Because of the wide-ranging specificity of ScFvpY, it is also able to be used for this purpose, and indeed is able to be used as a general-purpose detector reagent for detection of tyrosine phosphorylation in a wide variety of protein substrates.

In FIG. 3, yeast have been triply transformed with: (i) protein of interest, EGF receptor cytoplasmic tail fused to the AD domain of Gal4, (ii) detector protein, either the PTB domain of the adapter Shc, or ScFvpY, fused to the binding domain of LexA and (iii) the active kinase domain of tyrosine kinase Src or Csk, under the control of a methionine repressible promoter. Csk is another member of the Src family of tyrosine kinases, used as a control in this assay. In a screen of kinases these latter two would be replaced by expression of a cDNA library of tyrosine kinases. Yeast were then grown in the presence of methionine (1 mM), which acts to suppress expression of the kinase. For each assay three colonies are picked and grown, so as to triplicate data. In the presence of histidine (left hand panel), there is full growth of all colonies showing that when selection is removed all yeast colonies are able to grow. This demonstrates the non-toxicity of the AD and BD fusion constructs in these yeast. In the absence of histidine (right-hand panel), no growth would be predicted since methionine would suppress expression of the essential modifying enzyme (Src or Csk). No growth was seen in ShcPTB-BD transformants, however there was growth apparent in ScFvpY-BD transformants. This suggested leakiness of regulation of the HIS3 gene when ScFvpY is fused to the LexA DNA binding domain.

In order to overcome this problem, yeast were grown in the presence of the competitive inhibitor of histidine, 3-amino-1,2,4-triazole (3AT, 1 mM) (FIG. 4). Yeast were doubly transformed with EGFRt-AD and ScFvpY-BD and grown either in the presence or absence of histidine (1 mM), to allow selection. Interaction between the two fusion proteins is not expected in this experiment since there is no expression of a tyrosine kinase and therefore no means of permitting interaction and expression of HIS3 gene. In the presence of histidine growth is expected, and is seen (top left panel), thereby demonstrating viability of the yeast. In order to demonstrate the non-toxicity of the histidine antagonist 3AT, yeast were then grown in the presence of histidine (1 mM) and 25 mM 3AT, a supramaximal concentration (top right panel). Growth of yeast is maintained under this condition, demonstrating that even at 25 mM there is no toxic effects of 3AT on yeast viability. In the absence of histidine, as we have already shown, there is leakiness of the HIS3 gene in the absence of 3AT (bottom left panel), allowing growth of yeast. However, this growth is abolished in the presence of just 1 mM 3AT, thus demonstrating this to be a successful approach to overcoming leakiness of the HIS3 gene in the yeast transformed with the single-chain antibody detector protein ScFvpY-BD.

In order to demonstrate that yeast transformed with Src kinase domain construct would express a tyrosine kinase activity in a methionine-dependent manner, a dose-response relationship experiment was performed to detect tyrosine kinase activity. FIG. 5 shows a western blot of yeast whole cell lysates from yeast singly transformed with the Src kinase domain construct. Yeast were grown in media containing different concentrations of methionine as shown, and lysed into sample buffer. Proteins were run on SDS-PAGE 10% gel, transferred to PVDF membrane and blotted with 4G10 antiphosphotyrosine mAb. It may be seen that in the absence of methionine, Src is expressed and is able to phosphorylated several endogenous yeast proteins. Expression of Src is suppressed by concentrations of methionine of 0.2 mM and above, indicating the tight regulation of gene expression by the methionine regulated promoter. This clearly demonstrates expression of an active tyrosine kinase in the yeast.

Having determined optimal conditions for the tribrid assay, in terms of concentrations of 3AT required to prevent leakiness of the HIS3 gene in ScFvpY-BD transformed yeast, and in terms of the concentrations of methionine required to regulate expression of the tyrosine kinase, the inventors were able to perform a full tribrid experiment. In FIG. 6, yeast was triply transformed with: (i) protein of interest, EGF receptor cytoplasmic tail fused to the AD domain of Gal4, (ii) detector protein, either the PTB domain of the adapter Shc, or ScFvpY, fused to the binding domain of LexA and (iii) the active kinase domain of tyrosine kinase Src or Csk, under the control of a methionine repressible promoter. Data shown are in triplicate. Yeast were grown in the presence or absence of both methionine and histidine. In the presence of histidine there was full growth of yeast from all conditions, showing that expression of the AD and BD constructs was not toxic. In the absence of histidine, yeast grown in the presence of methionine, where expression of the tyrosine kinase is suppressed, showed no growth. However, in the absence of both histidine and methionine (far right panel), there was significant growth in yeast transformed with Src. The growth was more prominent when ScFvpY-BD was used as detector protein, rather than ShcPTB-BD. In addition, there was some selectivity for Src over Csk, since there was greater degree of yeast growth in yeast transformed with Src rather than Csk. All these experiments were conducted in the presence of 3AT (1 mM) to suppress leakiness of HIS3 gene. This experiment therefore demonstrates that, for EGFRt and ScFvpY partners, expression of an active kinase Src is able to promote interaction and therefore detection of phosphorylation of substrate. Selectivity is demonstrated such that Src is preferred as the upstream kinase over Csk, demonstrating the potential ability of the approach to screen cDNA libraries of kinases to identify kinases responsible for phosphorylating substrates. In a broader sense, this approach could then be extended to screen cDNA libraries, using ScFvs as detectors for post-translational modifications, to identify modifying enzymes of any description (including serine/threonine kinases, acetylases, glycosylases etc) for any substrate protein of interest.

FIG. 7 shows a difference set of kinases and substrates. Here LAT is used as substrate, fused either to Gal4 AD or LexA BD. Detector proteins are either PLCg2SH2 domains, fused to either Gal4 AD or LexA BD, or the ScFvpY this time fused to Gal4 AD. Appropriate double transformants of partners were made, as indicated in the figure. Yeast was then further transformed with the active kinase domains of either Src or Syk. Both these kinases have the potential for phosphorylating LAT on tyrosine in vivo. In this system it may be seen that, in the presence of histidine, expression of any these constructs is not toxic to the yeast since there is full growth under all conditions, whether in the presence or absence of methionine. In the absence of histidine however, there is no growth of yeast when expression of the tyrosine kinases is suppressed by methionine. However, when either Syk or Src is expressed, in the absence of methionine, there is full growth of yeast in every condition. This indicates that the tribrid assay is able to operate to detect interactions only when phosphorylation takes place, as described for FIG. 6 above.

It was possible however that the expressed tyrosine kinase may be acting as a bridge to couple the BD and AD fusion partners. In order to rule this possibility out, and in order to demonstrate that kinase activity is essential for the interaction to take place, the inventors generated a point mutant construct of Src kinase domain, as described in Methods. Yeast triply transformed with LAT-AD (as substrate protein), PLCγ2SH2-BD (as detector protein) and either the active kinase domain of Src or its inactive mutant, were grown in the presence or absence of methionine or histidine. Gene expression was monitored not only for HIS3 regulation of growth, but also for γ-galactosidase expression as described in Methods. FIG. 8 shows that yeast triply transformed with kinase active Src grow in media lacking histidine, only in the absence of methionine where expression of Src is switched on. This result is substantiated by expression of the other reporter gene, b-gal, which is expressed only in the absence of methionine also. In contrast however, the inactive point mutant of Src is not able to support AD and BD fusion protein interaction and switching on of reporter gene. This demonstrates that kinase activity is essential for the tribrid to operate and also that Src is not acting as a bridging protein for the AD and BD fusion proteins.

In summary then the inventors have validated, using two different enzyme/substrate partner systems, a method for screening a library of kinases, or other post-translational modifying enzymes. In order to detect the post-translational modification, a series of different detector proteins may be used. The most universally useful of these is a single-chain antibody directed, in this case, against phosphotyrosine. ScFvs may be generated directed against any post-translational modification, and are extremely versatile in this regard. This system may then be used to screen cDNA libraries of kinases or other modifying enzymes, which lie upstream of and regulate any substrate protein of interest.

Experimental Methods

Yeast Transformation

Saccharomyces cerevisiae strain TAT7 (MATa ade2, ura3, leu2, trp1, his1) was used for triple transformations. For histidine (HIS) auxotrophy assays, yeast transformants were spotted on the appropriate drop out plates lacking HIS and supplemented with 25 mM 3-aminotriazole (3-AT). β-galactosidase assays were performed on nitrocellulose filters as described below. General yeast methods were used as previously described.

β-Galactosidase Assays

The yeast transformants grown on the appropriate drop out plates containing HIS were replica plated onto nitrocellulose filters. The filters, yeast colonies containing side up, were dipped for 10-15 sec in liquid nitrogen, thawed at room temperature. The filters, yeast colonies side up, were then placed on Whatman 3MM filter papers soaked in Z-buffer (60 mM Na₂HPO₄, 40 mM NaH₂PO₄, 10 mM KCl, 1 mM Mg₂SO₄, pH-7.0) containing 0.3 mg/ml X-gal and 40 μM β-mercaptoethanol and incubated 37° C. 3-10 hrs for conversion of X-gal into a blue colour product.

Plasmid Constructs

The yeast-E. coli shuttle plasmid pLexA, which contains an ADH promoter and termination sequences, was used. Inserted into the polylinker is the gene for the E. coli protein LexA (amino acids 1-202) which binds to the Lex operator (BD) and restriction sites for cloning other genes in frame. This plasmid was digested with appropriate restriction enzymes and the following cDNAs were inserted to form ‘bait’ proteins: (i) human PLCy2 tandem SH2 domains, pPLCSH2, or (ii) Shc phosphotyrosine binding domain (Shc PTB), or (iii) a single chain antiphosphotyrosine antibody, ScFvpY, used a general detector to identify tyrosine phosphorylation multiple substrates. The prey protein substrate LAT or the C-terminal region of EGFR (EGFRt) was fused to the transcription activation domain (AD) for Gal4 to form plasmid pPrey. The tyrosine kinase domain (TK) of SRC, Syk and CSK were inserted into pRs426Met, which contains Met25 promoter to prevent gene expression in presence of methionine.

Generation of Dominant-Negative Mutant (DNM) of SRC

SRC was mutated at a critical residue (K298R) within the kinase domain to generate a kinase-null mutant. Standard site-directed mutagenesis was used to generate this mutant. The kinase was otherwise intact, but lacked any kinase activity and was therefore suitable for use as a dominant-negative mutant. The kinase domain of DNM SRC was cloned into pRS426Met.

Immunoblotting

Yeast cells (10⁷ cells/ml) were pelleted and broken by vigorous shaking in 0.2 ml of breaking buffer (100 mM Tris-HCl, pH 8, 1 mM DTT, 20% glycerol) after addition of glass beads. Proteins were separated by SDS-PAGE and transferred to PVDF blotting membranes. Membranes were incubated for 60 min at room temperature with phosphotyrosine primary antibody followed by secondary antibody and detected by ECL (Amersham, UK).

1. A screening method for regulatory enzymes, the method comprising the construction of tribrid cells containing genes encoding an expression library of putative enzymes, a bait protein or polypeptide fused to a known DNA binding domain and a prey protein which is an antibody that recognises a protein or polypeptide which has been post translationally modified, the prey protein being attached to a known protein active domain, whereby, in use, binding or recognition of the bait protein or polypeptide by the prey protein or polypeptide upon post-translational modification by an enzyme contained In the expression library causes transcription of a reporter gene or genes which allow recognition of the enzyme activity.

2. A method according to claim 1, in which the cell is a eukaryote.

3. A method according to claim 1 or claim 2, in which the cell is a yeast cell.

4. A method according to any one of claims 1 to 3, in which the enzyme is involved in the post-translational modification of nascent proteins or polypeptides.

5. A method according to claim 4, in which the enzyme is involved in the regulation of phosphorylation, glycosylation, sulphonation, acetylation side chain modification, nitrosylatlon, ubiquination, myristoylation or palmitoylation.

6. A method according to claim 4 or claim 5, in which the enzyme is a kinase or a phosphatase.

7. A method according to any preceding claim 1, in which the bait protein is an oncoprotein, a kinase, a phosphatase, a receptor protein, an adapter protein or a scaffolding protein.

8. A method according to any preceding claim, in which the prey protein is conformationally constrained within the cell.

9. A method according to claim 8, in which the prey protein is conformationally constrained by linkage of the carboxy and amino termini of the protein.

10. A method according to any preceding claim, in which the prey protein further comprises an epitope tag to enable rapid detection of fusion protein synthesis.

11. A tribrid cell for use in the method of claims 1 to 10, said tribrid cell being engineered to express an enzyme or putative enzyme from a cDNA library, a bait protein or polypeptide fused to a known DNA binding domain and a prey protein which is an antibody that recognizes a protein or polypeptide which has been post translationally modified, the prey protein being attached to a known protein active domain, whereby, in use, binding or recognition of the bait protein or polypeptide by the prey protein or polypeptide upon post-translational modification by the enzyme from the cDNA library causes transcription of a reporter gene or genes which allow recognition of the enzyme activity.

12. A tribrid cell according to claim 11, in which the cell is a eukaryote cell.

13. A cell according to claim 11 or claim 12, in which the cell is a yeast cell.

14. A method substantially as hereinbeore described with reference to and as illustrated by the Examples.

15. A cell substantially as hereinbefore described with reference to and as illustrated by the Examples.
